(19) United States
(12) Patent Application Publication
(10) Pub. No.: US 2017/0020379 A1
(43) Pub. Date: Jan. 26, 2017

(54) PROTECTIVE LARYNGOSCOPE HOUSING WITH LIGHT SOURCE STERILIZATION

(71) Applicants: Catherine Bell, Sausalito, CA (US); Chris Moore, San Francisco, CA (US)

(72) Inventors: Catherine Bell, Sausalito, CA (US); Chris Moore, San Francisco, CA (US)

(21) Appl. No.: 14/730,212

(22) Filed: Jun. 3, 2015

(51) Int. Cl. A61B 1/12; A61L 2/10; A61B 1/267

(52) U.S. Cl. CPC A61B 1/121; A61B 1/267; A61L 2/10

(57) ABSTRACT

In accordance with the teachings provided herein, a Protective Laryngoscope Housing With Light Source Sterilization can emit pulse UV light at a sufficiently short wavelength to cause Ultraviolet germicidal irradiation (UVGI) to maintain an easily accessible, yet sterile or germicidal environment for laryngoscope handles.

### RELATED APPLICATIONS

This application claims priority to U.S. Provisional Patent Application No. 62/007,411, filed Jun. 4, 2014, and entitled “Airway Bell” by Catherine Bell, MD and Chris Moore, CRNA, JD, which is incorporated herein by reference.

### BACKGROUND

Laryngoscopes and laryngoscope handles are essential tools for the practice of anesthesia. Laryngoscope handles are either rechargeable-battery or battery powered. The laryngoscope blades are attached to laryngoscope handles and are utilized for intubating patients. The blade, which is illuminated by power source contained in the handle, is inserted into the oropharynx, commonly known as the portion of the throat connected to the mouth. As such, the blade is exposed to potential contaminants such as bacteria and viruses. Since the blade and laryngoscope handle are used as a unit, they are both considered contaminated after use. Therefore, before re-use the laryngoscope handles and blades must be sterilized and this is done according to the manufacturers' recommendations.

Prior processes allowed for the laryngoscope handles and blades to be assembled and placed in a recharging dock, see FIG. 1. The re-charging station allows for a constant state of readiness as the laryngoscope handles are fully charged and the blade is attached. In the event of an anesthetic crisis, the required airway equipment is immediately available and ready for use, possibly saving time and lives as the anesthesia provider may establish a patent airway and breathe for the patient.

Recent changes to the standards for Operating Room sterile requirements have established a concern for the maintained sterility of the laryngoscope handles and blades. It is possible that the laryngoscope handles left exposed to the atmosphere of the operating room could become exposed to particulate matter from surgery. As the laryngoscope handles are not cleaned unless used in a case, those left in the charging station become vulnerable to OR environment contamination. The recommendation has been to keep laryngoscope handles and blades in sealed sterile packaging and only open them for a case. This process eliminates the state of readiness, which is essential for the practice of safe anesthesia care.

The foregoing examples of the related art and limitations related therewith are intended to be illustrative and not exclusive. Other limitations of the related art will become apparent upon a reading of the specification and a study of the drawings.

### SUMMARY

The following examples and aspects thereof are described and illustrated in conjunction with systems, tools, and methods that are meant to be exemplary and illustrative, not limiting in scope. In various examples, one or more of the above-described problems have been reduced or eliminated, while other examples are directed to other improvements.

According to these teachings, sterilization of laryngoscope handles involves a housing as illustrated, with a light source. The function of the light source may vary. Multiple light sources can be used. A light source can provide a germicidal environment, and another can provide sterilization. An exemplary embodiment is provided which includes a protective housing equipped with doors, a light source based sterilization technology and a laryngoscope handle re-charging station.

Advantageously, the combination of sterilization technology and a re-charging station within a single enclosed housing allow laryngoscope handles and blades can be kept in an easily accessible yet sterile environment without compromising the readiness of the equipment.

### BRIEF DESCRIPTION OF THE DRAWINGS

FIG. 1 depicts a front view of an example of protective housing for a laryngoscope handle charger.

FIG. 2 depicts a rear view of an example of protective housing for a laryngoscope handle charger.

FIG. 3 depicts an exploded front view of an example of protective housing for a laryngoscope handle charger.

### DETAILED DESCRIPTION

in the following description, several specific details are presented to provide a thorough understanding. One skilled in the relevant art will recognize, however, that the concepts and techniques disclosed herein can be practiced without one or more of the specific details, or in combination with other components, etc. In other instances, well-known implementations or operations are not shown or described in detail to avoid obscuring aspects of various examples disclosed herein.

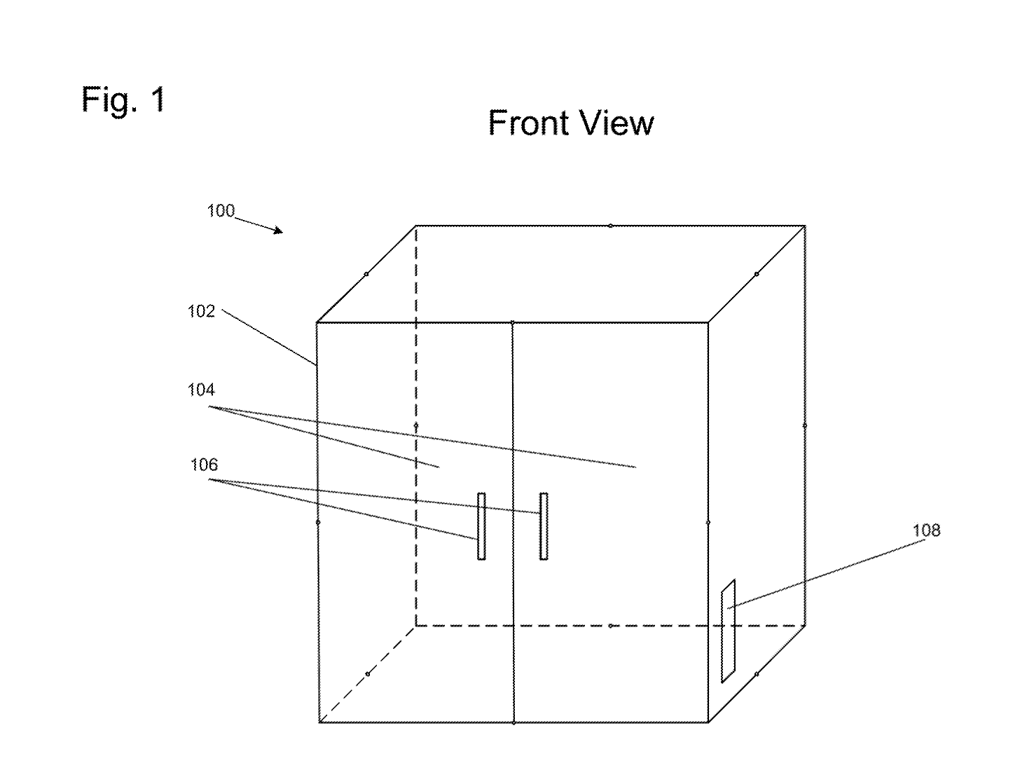

FIG. 1 depicts an example of a front view of a protective housing for a laryngoscope handle charger 100. In the example of FIG. 1, the device 100 includes a housing 102, two doors 104 in the front of the housing, two door handles 106, one affixed to each door, and a ready light 108 which illuminates when the device has completed the UV irradiation cycle.

In the example of FIG. 1, the housing 102 is a box that can be constructed of stainless steel, plastic or any other known or convenient material; it is sealed at all joints and can fit over an existing re-charging station for recharging the laryngoscope handles. Interior insulation can be included to minimize transmission of heat to the exterior of the housing. The dimensions of the housing can be increased to allow for the storage of additional laryngoscope blades of various sizes to be suspended from the interior panel adjacent to the recharging station. The additional blades would have full exposure to the UV light thus allowing them to remain in a bacteriostatic condition and immediately available for use.

In the example of FIG. 1, the two doors 104 in the front of the housing can be constructed of the same material as the housing, which allow for a laryngoscope handle (which has been sterilized according to manufacturers' recommendation) to be placed into the housing, and the doors closed, thus preventing further exposure to the atmosphere of the operating room. The blade may also be attached to the laryngoscope handle and kept in the housing as it will also be in a state of continued sterilization and readiness. In addition to preventing contamination from the atmosphere, the housing contains a sterilization technology which ensures continued sterile equipment ready to use at any time.

In the example of FIG. 1, a door handle 106 is affixed to each door to allow the doors to be easily opened and closed.

In the example of FIG. 1, the ready light 108 illuminates when the device has completed the sterilization cycle so that the provider is assured that the laryngoscope is sterile and ready for use.

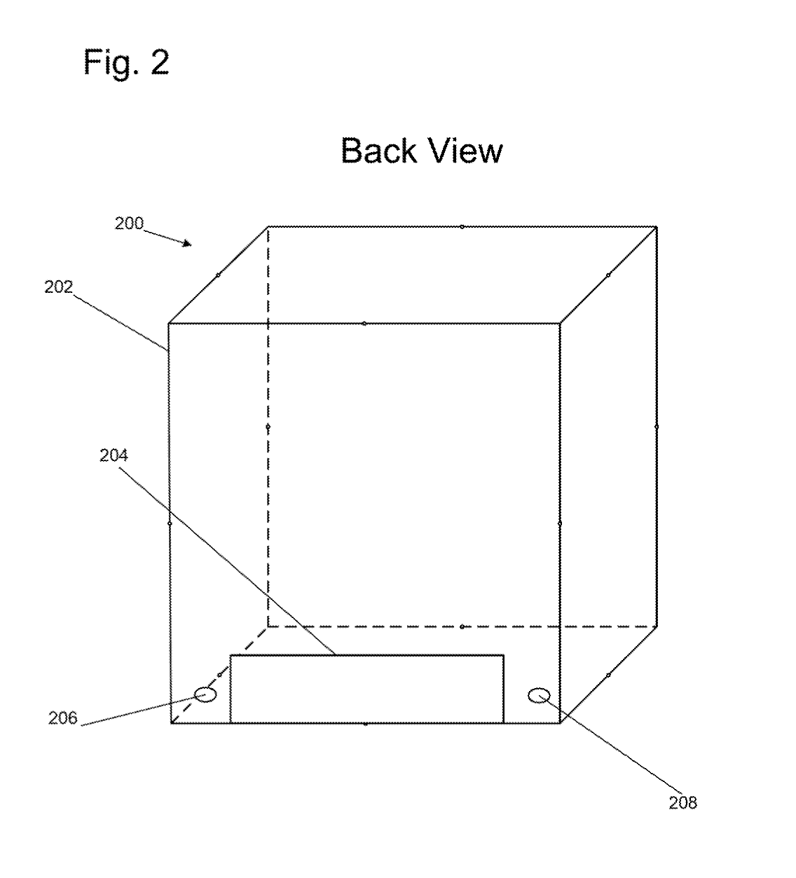

FIG. 2 depicts an example of a back view of a protective housing for a laryngoscope handle charger 200. In the example of FIG. 2, the protective housing for a laryngoscope charger 200 includes a housing 202, a trap door for outlets 204, a possible outlet for a power cord to the UV light 206, and a possible outlet for a power cord to the charger or outlet for a single, unified power cord 208.

In the example of FIG. 2, the housing 202 can be as described above.

In the example of FIG. 2, the trap door for outlets 204 can be constructed of the same material as the housing; which allows for outlet cables to run from the charging station and UV light source inside the housing to an electric power source external to the housing.

In the example of FIG. 2, the possible outlet for a power cord to the UV light 206 can be composed of a variety of materials, including but not limited to plastic, glass, acrylic, rubber, resin, or another known or convenient material, and which can provide an outlet for a power cord for a UV light source while also minimizing the unnecessary leakage of UV light from the housing.

In the example of FIG. 2, the possible outlet for a power cord to the charger or outlet for a single unified power cord 208 can be composed of a variety of materials, including but not limited to plastic, glass, acrylic, rubber, resin, or another known or convenient material, and which can provide an outlet for a power cord for a laryngoscope re-charging station while also minimizing the unnecessary leakage of UV light from the housing. Future models would contain designs for a built-in recharging station or a plug external to the housing for the existing electrical attachment so that the unit would only require a single electrical outlet in the operating room or procedure room.

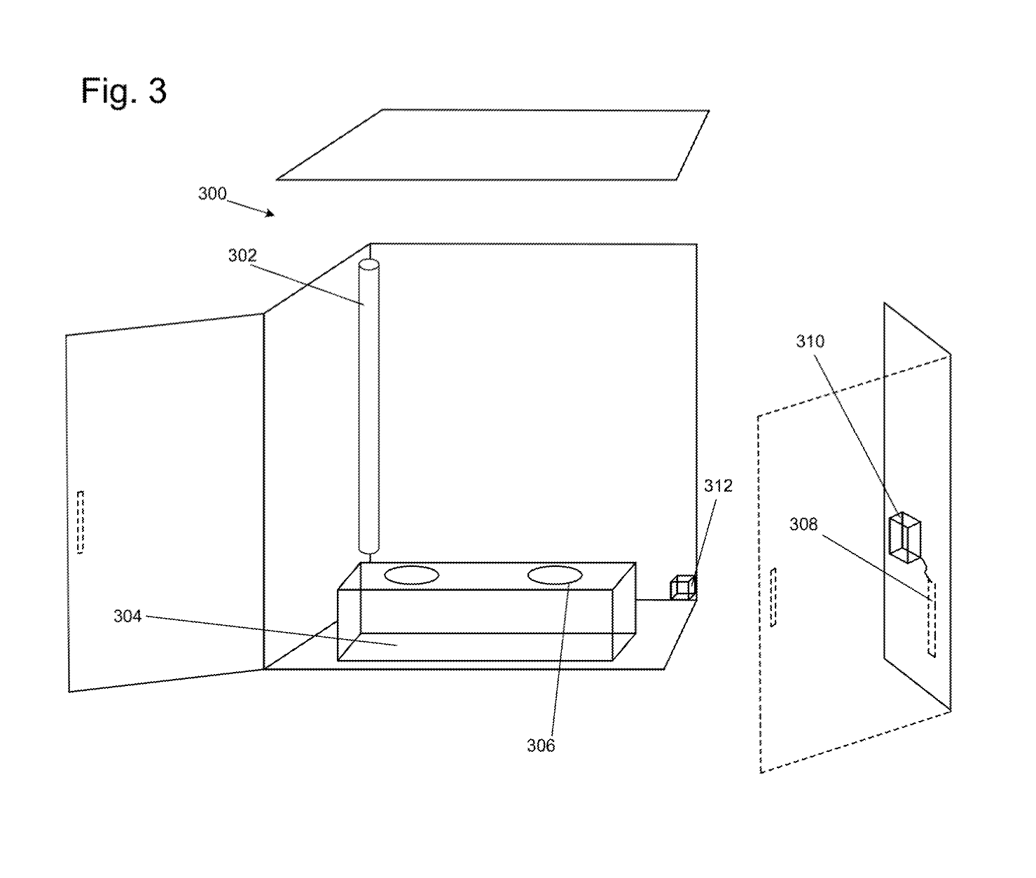

FIG. 3 depicts an example of an exploded view of a protective housing for a laryngoscope handle charger 300. In the example of FIG. 3, the protective housing for a laryngoscope charger 300 includes a UV light source 302, a re-charging station 304, laryngoscope handle slots 306 in the re-charging station, a ready light 308, a sterilization sensor 310, and internal control mechanics for a light source trigger 312.

Ultraviolet germicidal irradiation (UVGI) is a disinfection method that uses ultraviolet (UV) light at sufficiently short wavelength to kill microorganisms. UVGI utilizes short-wavelength ultraviolet radiation that is harmful to microorganisms. It is effective in destroying the nucleic acids in these organisms so that their DNA is disrupted by the UV radiation, leaving them unable to perform vital cellular functions.

In the example of FIG. 3, the UV light source 302 emits pulse UV light at a sufficiently short wavelength to cause UVGI. This UVGI light source is obtained under separate patent and is separately manufactured and then mounted in the housing. When the housing is opened, a laryngoscope handle is inserted into the charger and the door closed, housing the UVGI process is initiated, germicidal inactivation and/or sterilization occurs and any contamination from the atmosphere is prevented.

In the example of FIG. 3, the re-charging station 304 is a standard laryngoscope re-charging station.

In the example of FIG. 3, the laryngoscope handle slots 306, as per the specifications of the laryngoscope re-charging station, are designed appropriately for the laryngoscope(s) to be charged.

In the example of FIG. 3, the external ready light 308 can be as described in above.

In the example of FIG. 3, the sterilization sensor 310, can be a timing device measuring the duration of time the laryngoscope handles have been exposed to UV light to determine when the laryngoscope handles are sterilized. Sterilization sensor 310 is connected to the ready light so that when the handles are sterilized, the ready light illuminates.

In the example of FIG. 3, the internal control mechanics for a light source trigger 312, cause the UV light source to be triggered to emit pulse UVGI when the housing door is opened, a handle is inserted into the re-charging station and the housing door is closed. Conversely, when the doors to the housing are opened, the trigger 310 can automatically switch off the ultraviolet light source so that the health care providers near the unit will not be exposed to the ultraviolet light and the operating room or procedure will not be unnecessarily illuminated when doctors and other practitioners are accessing the laryngoscope handles. Once a laryngoscope handle is placed into the housing the UVGI process is initiated, germicidal inactivation and/or sterilization occurs and any contamination from the atmosphere is prevented.

It will be appreciated to those skilled in the art that the preceding examples and embodiments are exemplary and not limiting to the scope of the present invention. It is intended that all permutations, enhancements, equivalents, and improvements thereto that are apparent to those skilled in the art upon a reading of the specification and a study of the drawings are included within the true spirit and scope of the present invention. It is therefore intended that the following appended claims include all such modifications, permutations and equivalents as fall within the true spirit and scope of the present invention.

What is claimed is:

1. A laryngoscope handle charging station comprising,
a housing including a laryngoscope handle charger,
a UV light source operable to emit UV light at a wavelength effecting Ultraviolet germicidal irradiation (UVGI); and
a sterilization sensor operable to trigger a ready light upon completion of the UV irradiation cycle;

2. The laryngoscope handle charging station of claim 1, wherein the housing is a box constructed of stainless steel, sealed at all joints, and able to enclose laryngoscope handles with blades affixed.

3. The laryngoscope handle charging station of claim 1, further comprising two doors in the front of the housing sufficiently large enough to allow for a laryngoscope handle with blade attached to be placed into the housing and the doors closed.

4. The laryngoscope handle charging station of claim 1, further comprising a trap door which allows for outlet cables to run from the charging station and UV light source inside the housing to an electric power source external to the housing.

5. The laryngoscope handle charging station of claim 1, further comprising a sealed outlet port for a UV light source power cord to preventing contamination of the sterilization process.

6. The laryngoscope handle charging station of claim 1, further comprising an outlet port for a laryngoscope handle charger power cord which allows the laryngoscope handle charger power cord to exit the housing while preventing contamination of the sterilization process.

7. The laryngoscope handle charging station of claim 1, further comprising an outlet port for a single power cord for the UV light source and the laryngoscope handle charger which allows the single power cord to exit the housing while preventing contamination of the sterilization process.
